(19) United States
(12) Patent Application Publication
(10) Pub. No.: US 2005/0123612 A1
(43) Pub. Date: Jun. 9, 2005

(54) Ribavirin granulate for producing coated tablets

(76) Inventors: Cornelis Sobel, Mainz (DE); Gerald Huber, Pluderhausen (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/499,783

(22) PCT Filed: Dec. 21, 2001
(86) PCT No.: PCT/EP01/15202

(51) Int. Cl. A61K 31/7056; B29B 9/00; A61K 38/21

(52) U.S. Cl. 424/489; 514/43; 264/5; 424/85.7; 264/109

(57) ABSTRACT

The present invention relates to a method for the production of a ribavirin-containing granulate, comprising the production of a granulate solution, comprising mixing of a binding agent with an isopropanol/water- or ethanol/water-mixture, stirring of the granulate solution into a mixture of ribavirin-powder with a hydrophilic or swellable solid adjuvant, and sieving and drying the so-obtained granulate, which is characterised in that the portion of isopropanol or ethanol of the alcohol/water-mixture is between 65% to 85% by weight. Based on this granulate, a ribavirin-tablet can be produced that can be used for the treatment of diseases, such as HCV.

The invention relates to a ribavirin-granulate for the production of ribavirin-containing tablets and their uses.

The international non-proprietary name “ribavirin” (1-β-D-Ribafuranosyl-1,2,4-triazole-3-carboxamide) designates a nucleoside analogue which is approved as virusstatic with limited indications in the therapy of humans. Due to its chemically modified ribose moiety, ribavirin exhibits a typical feature of an anti-metabolite or its precursor, respectively. Already described is an inhibition of the synthesis of guanosine-nucleosides by ribavirin, an inhibition of the RNA-polymerase and an indirect inhibition of protein biosynthesis. The particular mechanism of action of ribavirin, nevertheless, is still not resolved.

In principle, ribavirin is active against a broad spectrum of viruses (e.g. hepatitis, influenza, measles, herpes, AIDS). Therapeutically relevant are currently only its activities against the respiratory-syncytial-virus (RSV) and the hepatitis C-virus (HCV).

In Germany, ribavirin was initially approved exclusively for aerosol therapy of most severe bronchopulmonal infections caused by RSV. A ribavirin-containing dry substance for the production of solutions for inhalation is available since 1993 under the trademark “Virazole®” (ICN).

In the meantime, ribavirin was approved as “Rebetol®” (Essex Pharma) in peroral application forms for the therapy of chronic hepatitis C, in combination with the cytokine interferon α-2b (“Intron A®”, Essex Pharma). This combined therapy is successfully used in non-pretreated patients with histologically proven HCV-infection, especially in cases of relapse patients with chronic hepatitis C, which earlier responded to interferon α.

The only peroral application form of ribavirin available so far, namely Rebetol®, are hard capsules on the basis of gelatine.

From a pharmaceutical point of view, capsules exhibit a series of disadvantages. The accuracy by which the active agent can be dosed is lower in the case of capsules in comparison to e.g. tablets. Accordingly, the legally highest admissible variance of the mass per dosage unit is ±7.5% for capsules and ±5% for tablets. The patient should swallow the capsule preferably intact, which can be problematic for certain patients (children, elderly people, seriously ill people) and could, in some instances, influence the willingness for treatment (“compliance”). The release of the active compound then takes place concomitantly with the rapid degradation of the capsule in the stomach. A controlled release over time is practically not possible.

The packaging of medical compounds into capsules in addition exhibits also practical disadvantages. A production of different dosage units in a homologous series is technically much more laborious, as e.g. in tablets. A flexible dosage by simply breaking apart is also impossible in the case of capsules. A further critical feature that became more important in recent times is that capsules contain parts of animal origin, such as gelatine, which includes the risk of transferring the causing agent of, for example, BSE.

It could be shown in animal experiments that ribavirin is teratogenic in particular in the first six weeks of embryo development. This leads to the fact that female care personnel must be protected from an exposition and, furthermore, that the participation of women in the production of ribavirin-containing medication has to be excluded. When sticking together the capsules, it is unavoidable that ribavirin powder will stick to its outer surface. In contrast to tablets, gelatine capsules can not be coated with an aqueous lacquer, that would be suited to firmly incorporate the powder. Not only during packaging of capsules, but also during the application, e.g. by care taking personnel, it is therefore highly likely for the personnel to get in contact with ribavirin. Finally, the capsules that are more or less loosely stuck together are mechanically not very stable, which causes an additional risk when handling, and in particular during transport.

In addition to the general disadvantages of a capsule, therefore in the case of ribavirin another peroral application form is particularly desirable.

It is therefore an object of the present invention to provide ribavirin in the application form of a tablet, in particular a film tablet.

According to the invention, this object is solved by providing a method for the production of a ribavirin-containing granulate, comprising:
producing a granulate solution, comprising mixing of a binding agent with an isopropanol/water- or ethanol/water-mixture,
stirring of said granulate solution into a mixture of ribavirin-powder together with a hydrophilic or swellable solid adjuvant, and
sieving and drying the granulate thus obtained, characterised in that the portion of isopropanol or ethanol of the alcohol/water-mixture is between 65% to 85% by weight, preferably between 75% to 85% by weight when using ethanol and between 65% to 75% by weight when using isopropanol.

In a preferred embodiment, the binding agent is selected from cellulose and/or derivatives thereof, or starch and/or derivatives thereof. Particularly preferred is polyvinylpyrrolidone, in particular polyvidone with a molecular weight of less than 90,000, such as K25.

In a preferred method, the hydrophilic or swellable solid adjuvant is selected from microcrystalline cellulose, sugar alcohols, starch (e.g. corn starch) and/or derivatives thereof, or sugars (e.g. lactose) and/or derivatives thereof.

In another preferred method, the humid granulate is sieved with a sieve of ≦5 mm, preferably 2 mm. Thereby, the granulate will become homogenous, but is nevertheless still too crude for further processing.

In another preferred method, the granulate is dried to a residual humidity (measured at 70° C.) of less than 3%.

In a further preferred method, the dried granulate is finally sieved with a sieve of ≦2 mm, preferably 1 mm. By this treatment, the unwanted portion of dust is critically minimised and for the following process steps the distribution of the particle sizes is optimised.

Particularly preferred is the use of the granulate for the production of a ribavirin-containing tablet, wherein the method for production comprises:
mixing of the ribavirin-granulate with microcrystalline cellulose, blasting agent and highly disperse siliciumdioxide and/or talcum and/or calcium hydrogen phosphate,
addition of a lubricant, such as magnesium stearate, fumaric acid, adipinic acid or PEG, and
tabletting of said mixture.

In a preferred method for the production of a ribavirin-tablet, the blasting agent is cross-linked polyvinylpyrrolidone, in particular crospovidone or another adjuvant that is swellable in water.

A particularly preferred method for the production of a ribavirin-tablet comprises the application of a film coating.

In an particularly preferred method for the production of a ribavirin-tablet the film coating comprises titanium dioxide or another suitable pigment; isopropanol, ethanol or water or mixtures thereof; and a film forming agent, such as hydroxypropylmethyl cellulose, hydroxypropylmethyl cellulose phthalate, ethyl cellulose, polyacrylates or shellac and derivatives thereof.

The above-indicated adjuvants and additives for the production of the granulate according to the invention, and the tablet according to the invention represent preferred embodiments of these compounds. It is readily apparent to the skilled person in this technical field that these compounds can be replaced or supplemented with other compounds that have the same properties without departing from the general concept of the present invention. Suitable other compounds can be derived by the skilled person in particular from the following standard literature: Pharmazeutische Technologie: hrsg. von Heinz Sucker, bearb. von H. Asche—2., neu bearb. Aufl.—Stuttgart; New York: Thieme, 1991.—XXII, 801 S.; ISBN 3-13-395802-X; Pharmazeutische Technologie: Kurt H. Bauer; Karl-Heinz Frömming; Claus Führer. Unter Mitarb. von Engelbert Graf—5., überarb. Aufl.—Stuttgart; Jena; Lübeck; Ulm: Fischer; Frankfurt [Main]: Govi-Verl., 1997.—XV, 472 S.; ISBN 3-437-25630-0, 3-7741-0638-X; Ab 6. Aufl. u.d.T.: Lehrbuch der pharmazeutischen Technologie; Pharmazeutische Technologie: moderne Arzneiformen: Lehrbuch für Studierende der Pharmazie, Nachschlagewerk für Apotheker in Offizin, Krankenhaus und Forschung; 72 Tabellen/von Rainer H. Müller und Gesine E. Hildebrand. Mit Beitr. von: K. H. Bauer—2., durchges. und erw. Aufl.—Stuttgart: WVG, Wiss. Verl.-Ges., 1998.—XVII, 471 S.: Ill.; (dt.); ISBN 3-8047-1549-4; Pharmazeutische Technologie: industrielle Herstellung und Entwicklung von Arzneimitteln; Ingfried Zimmermann.—Berlin; Heidelberg; New York; Barcelona; Budapest; Hon*: Springer, 1998.—XX, 644 S.; ISBN 3-540-63944-6; Pharmazeutische Technologie: für Studium und Beruf; von Rudolf Voigt.—9., völlig überarb. Aufl./bearb. von Alfred Fahr.—Stuttgart: Dt. Apotheker-Verl., 2000.—XXXII, 687 S.; (Wissen & Praxis); ISBN 3-7692-2649-6; bis zur 8. Aufl. erschienen im Verlag Ullstein Mosby Wiesbaden; Literaturverzeichnis S. [649]-651; Arzneiformenlehre: ein Lehrbuch der Galenik für Theorie und Praxis; mit 44 Tabellen/von Ursula Schöffling.—3., völlig neu bearb. und erw. Aufl.—Stuttgart: Dt. Apotheker-Verl., 1998.-464 S.; ISBN 3-7692-2254-7; Propädeutikum der Arzneiformenlehre/Claus-Dieter Herzfeldt.—2. Aufl.—Berlin; Heidelberg: Springer, 2000.—XVI, 249 S.; Galenik/Claus-Dieter Herzfeldt; 1; (Springer-Lehrbuch); ISBN 3-540-65265-5; Grundlagen der Arzneiformenlehre Claus-Dieter Herzfeldt . . . (Hrsg.).—Berlin; Heidelberg: Springer, 1999.—XV, 618 S.; Galenik/Claus-Dieter Herzfeldt; 2; (Springer-Lehrbuch); ISBN 3-540-65291-4; Propädeutische Arzneiformenlehre: Einführung in die Arzneiformenherstellung in der Apotheke; von Engelbert Graf u. Christian Beyer.—3., völlig neu bearb. Aufl. von Christian Beyer.—Stuttgart: Wissenschaftliche Verlagsgesellschaft, 1993.—206 S.; ISBN 3-8047-1267-3; Physikalische Pharmazie: pharmazeutische angewandte physikalisch-chemische Grundlagen; Martin; Swarbrick; Cammarata. Hrsg. u. vollst. überarb. von H. Stricker.—3., völlig neu bearb. u. erw. Aufl.—Stuttgart: Wiss. Verl.-Ges., 1987.—XVI, 587 S.; ISBN 3-8047-0893-5; Original: Physical pharmacy; und Lexikon der Hilfsstoffe für Pharmazie, Kosmetik und angrenzende Gebiete/Herbert P. Fiedler.—Aulendorf: Ed. Cantor; Der pharmazeutische Betrieb; Bd. 9; ISBN 3-87193-101-2, 3-87193-173-X Lexikon der Hilfsstoffe für Pharmazie, Kosmetik und angrenzende Gebiete/Herbert P. Fiedler);—3., überarb. und erw. Aufl.—1989.

Preferred is a ribavirin-containing tablet that is produced according to the method of the invention.

Particularly preferred is the therapeutical use of the ribavirin-tablet for the treatment of diseases as indicated, in particular viral diseases, such as HCV. Furthermore, the therapy can be performed in combination with a cytokine, in particular an interferon, such as interferon α and derivatives thereof.

The portion of the isopropanol/ethanol of the granulate solution, in which the ribavirin-powder is included during production, was identified as being essential for the structure and flow characteristics of the granulate according to the invention. If this portion is to high, the granulate will become mechanically unstable and has a bad capability of flow. In turn, in case of a too high portion of water, the granulate will become too hard and brittle to be compressed. In both cases, in particular the unwanted portion of dust will sharply increase. Surprisingly, an approximate 80% portion of ethanol in case of an ethanol/water-mixture or an approximate 70% portion of isopropanol in case of an isopropanol/water-mixture proved as optimal.

The good flow characteristics of the granulate substantially contribute to meeting the provided ribavirin-dosage per tablet as exactly as possible, which is for particular relevance in view of the stipulated security of a medication. The mean amount of drug in the medication form varies by approximately ±3%, which leads to an deviation that is lower as legally admissible (±5%), and essentially lower as admissible in the case of capsules. Furthermore, due to the flow characteristics, the portion of waste that is produced will be low. The production of different dosage units in a homologous series in case of a defined composition of the granulate will also be easily possible by modifying the weight of the individual tablet accordingly.

One essential advantage of the ribavirin-tablet is furthermore the fact that it can be coated with a water soluble film. By applying such a film, the ribavirin-dust that is potentially present will be bound and the tablet will be sealed. Accordingly, staff and the environment will be better protected from the teratogenic ribavirin. Furthermore, a certain control of time of the ribavirin-release in the stomach will be possible via the film tablet.

In the following, the advantageous properties of the invention will be further explained based on two embodiments:

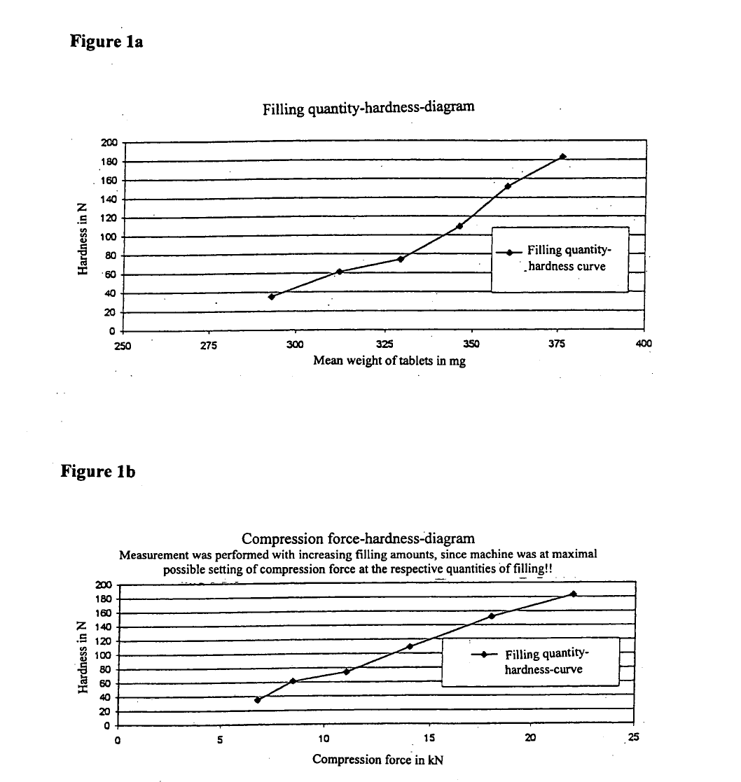

FIG. 1: Hardness of ribavirin-tablets as result of the compression force at tabletting

FIG. 1a: filling quantity-hardness-diagram

FIG. 1b: compression force-hardness-diagram

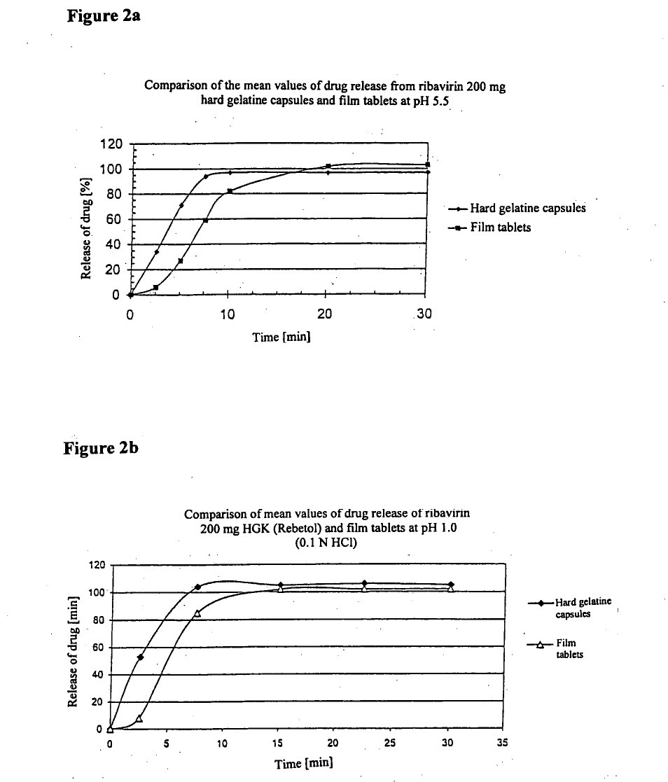

FIG. 2: Release kinetics of ribavirin: Comparison of ribavirin-tablets with hard gelatine capsules

FIG. 2a: Comparison of the release kinetics of ribavirin (200 mg) from hard gelatine capsules (Rebetol®-sample) and film tablets at pH 5.5 (mouth) (phosphate buffer). Indicated are mean values of three experiments.

FIG. 2b: Comparison of the release kinetics of ribavirin (200 mg) from hard gelatine capsules (Rebetol®-sample) and film tablets at pH 1.0 (stomach) (0.1 N HCl). Indicated are mean values of six experiments.

FIG. 1 represents the connection between the compression force that acts during tabletting of the granulate and the hardness of the resulting ribavirin-tablets at a diameter of 10.4 mm.

The force that is used during compression of the final mix-ribavirin will be maximal at a filling quantity of 300 mg per dosage unit and can not be further increased due to technical reasons. Therefore, an increase of the compression force was simulated by increasing the filling quantity (FIG. 1a). It was found that the hardness of the tablet as obtained increased with the mean weight of the tablets, namely from 35 N at 293 mg to 152 N at 360 mg. With 376 mg tablet weight, finally a borderline value was reached, above which good tabletting properties could no longer be ensured.

If the hardness of tablets is put in relation to the compression force (FIG. 1b), the achieved hardnesses will correspond to a compression force between 6.8 kN (at 35 N hardness) and 18 kN (at 152 N hardness).

Due to the advantageous structure of the granulate, a compression force of a mean value (6.8-8.5 kN) was sufficient in order to produce tablets with a sufficient hardness (35-61 N) (see FIG. 1). Since only moderate heat is produced, the drug will be exposed to a correspondingly lower thermal stress.

FIG. 2 shows the release kinetics of the tablets according to the invention in vitro. Due to the slightly delayed release, the medication security of the tablet of the present invention will be additionally increased, in comparison to the present capsules, since a certain time passes between swallowing (tablet in mouth) and the release (tablet in stomach). This effect, in addition, is independent from the dosage and can also be achieved with small tablet sizes. In contrast, ribavirin will be immediately released from hard gelatine capsules under weak acidic conditions (FIG. 2a) as present in the oral cavity, as well as at the pH-value according to the gastric juice (FIG. 2b), which can be problematic for the medication security.

In the following, two embodiments of the invention are presented:

### EXAMPLE 1

### In one embodiment, one tablet contains 200 mg±3% ribavirin in addition to additives, according to the recipe:

| | |
|---|---|
| ribavirin | 200.00 mg |
| polyvidone K25 | 16.00 mg |
| microcrystalline cellulose | 77.00 mg |
| crospovidone | 3.50 mg |
| silicium oxide | 2.00 mg |
| magnesium stearate | 1.50 mg |
| in total | 300.00 mg |

### EXAMPLE 2

In a particularly preferred embodiment, the tablet of the above-described recipe is additionally coated with a water soluble film (“lacquer”, “coating”), which consists of:

| | |
|---|---|
| hydroxypropylmethyl cellulose | 4.00 mg |
| titanium dioxide | 2.00 mg |
| | 6.00 mg |
| or, alternatively, consists of: | |
| hydroxypropylmethyl cellulose | 4.00 mg |
| titanium dioxide | 2.00 mg |
| polyethylenglycol 6000 | 1.00 mg |
| (Macrogol 6000) | |
| | 7.00 mg |

In the following, the production of a batch of ribavirin-tablets in a production scale is exemplified:

All compounds used in this method are in compliance with the limitations of the Arzneimittelgesetz. The same applies to the apparatuses as used.

1. Production of Ribavirin-Granulate

1.1 Premix I (Granulate Solution):

The granulate solution consists of polyvinylpyrrolidone (polyvidone K25, kollidone 25, purchased from BASF, Ludwigshafen) (2.00 kg) that was completely dissolved by stirring in a mixture of ethanol 96% (0.02 kg) and water (3.25 kg) (total weight: 14.27 kg).

1.2 Premix II (Granulate):

Ribavirin (36.3 kg) and microcrystalline cellulose (Avicel PH101, purchased from Lehmann & Voss, Hamburg) (6 kg) are first mixed and then subsequently processed to a homogenous granulate by the addition of the granulate solution (14,27 kg; time for addition approximately 10 minutes). The granulate is sieved in its humid state through a 2.00 mm Frewitt sieve, and is dried for approximately 3.5 hours at 60° C., up to a residual humidity of less than 3% (measuring temperature of 70° C.). Finally, the dried granulate is sieved through a 1.0 Frewitt sieve in order to achieve a consistent and low-dust granulate structure.

2. Production of the Ribavirin Final Mixture

To a mixture of microcrystalline cellulose (2.66 kg), polyvinylpyrrolidone (cropovidone, kollidone CL, obtained from BASF) (0.212 kg), high disperse silicium dioxide (Aerosil 200, obtained from Degussa) (0.121 kg), and polyvidone K25 (0.030 kg), ribavirin-granulate (14.767 kg) is admixed. Magnesium stearate (0.091 kg) is added to this mixture, and it is mixed again.

3. Tabletting

The ribavirin final mixture is compressed by a machine in portions of about 300 mg (corresponding to 200 mg ribavirin) to form bi-convex tablets of about 4.5-5.5 mm height and a diameter of about 10±1.2 mm, preferably 9.0-9.7 mm.

4. Film Coating

The coating of the tablet cores is performed by spray-application of a mixture of titanium dioxide (0.196 kg), suspended in water (0.5 kg), and hydroxypropylmethyl cellulose (Pharmacoat 606, obtained from Synthapharm) (0.392 kg), which has been completely dissolved in water (2.5 kg) before.

1. A method for the production of a ribavirin-containing granulate, comprising
producing a granulate solution, comprising mixing of a binding agent with an isopropanol/water- or ethanol/water-mixture,
stirring of said granulate solution into a mixture of ribavirin-powder with a hydrophilic or swellable solid adjuvant, and
sieving and drying the granulate thus obtained,
characterised in that the portion of the isopropanol or ethanol of the alcohol/water-mixture is from 65% to 85% by weight.

2. The method for the production of a ribavirin-containing granulate according to claim 1, characterised in that the portion of the alcohol/water-mixture is from 75% to 85% by weight when using ethanol and from 65% to 75% by weight when using isopropanol.

3. The method for the production of a ribavirin-containing granulate according to claim 1, characterised in that the binding agent is cellulose and/or derivatives thereof or starch and/or derivatives thereof.

4. The method for the production of a ribavirin-containing granulate according to claim 1, characterised in that the hydrophilic or swellable solid adjuvant is selected from microcrystalline cellulose, sugar alcohols, starch and/or derivatives thereof or sugars and/or derivatives thereof.

5. The method for the production of a ribavirin-containing granulate according to claim 1, characterised in that the humid granulate is sieved with a sieve of ≦5 mm.

6. The method for the production of a ribavirin-containing granulate according to claim 1, characterised in that the granulate is dried to residual humidity of less than 3%. (measuring temperature of 70° C.).

7. The method for the production of a ribavirin-containing granulate according to claim 1, further comprising a final sieving of the dried granulate with a sieve of ≦2 mm.

8. A method for the production of a ribavirin-tablet, wherein said method comprises forming a tablet from a ribavirin-granulate wherein said granulate is produced by a method comprising
producing a granulate solution, comprising mixing of a binding agent with an isopropanol/water- or ethanol/water-mixture,
stirring of said granulate solution into a mixture of ribavirin-powder with a hydrophilic or swellable solid adjuvant, and
sieving and drying the granulate thus obtained,
characterised in that the portion of the isopropanol or ethanol of the alcohol/water-mixture is from 65% to 85% by weight.

9. A method for the production of a ribavirin-containing tablet, comprising
A. preparing a ribavirin-granulate by a method comprising
producing a granulate solution, comprising mixing of a binding agent with an isopropanol/water- or ethanol/water-mixture.
stirring of said granulate solution into a mixture of ribavirin-powder with a hydrophilic or swellable solid adjuvant, and
sieving and drying the granulate thus obtained,
characterised in that the portion of the isopropanol or ethanol of the alcohol/water-mixture is from 65% to 85% by weight
B. mixing of the ribavirin-granulate with microcrystalline cellulose, blasting agent and highly disperse siliciumdioxide and/or talcum and/or calciumhydrogen phosphate,
C. adding a lubricant, and
D. tabletting of said mixture.

10. The method for the production of a ribavirin-containing tablet according to claim 9, characterised in that the blasting agent is a cross-linked polyvinylpyrrolidone.

11. The method for the production of a ribavirin-containing tablet according to claim 9, further comprising the application of a film coating.

12. The method for the production of a ribavirin-containing tablet according to claim 11, characterised in that the film coating comprises titanium dioxide or another suitable pigment; isopropanol, ethanol, water or mixtures thereof; and a film-forming agent.

13. A ribavirin-containing tablet, produced by:
A. preparing a ribavirin-granulate by a method comprising
producing a granulate solution, comprising mixing of a binding agent with an isopropanol/water- or ethanol/water-mixture,
stirring of said granulate solution into a mixture of ribavirin-powder with a hydrophilic or swellable solid adjuvant, and
sieving and drying the granulate thus obtained,
characterised in that the portion of the isopropanol or ethanol of the alcohol/water-mixture is from 65% to 85% by weight
B. mixing of the ribavirin-granulate with microcrystalline cellulose, blasting agent and highly disperse siliciumdioxide and/or talcum and/or calciumhydrogen phosphate,
C. adding a lubricant, and
D. tabletting of said mixture.

14. A method for the treatment of disease wherein said method comprises administering, to a patient in need of such treatment, a ribavirin-containing tablet produced by:
A. preparing a ribavirin-granulate by a method comprising
producing a granulate solution, comprising mixing of a binding agent with an isopropanol/water- or ethanol/water-mixture,
stirring of said granulate solution into a mixture of ribavirin-powder with a hydrophilic or swellable solid adjuvant, and
sieving and drying the granulate thus obtained,
characterised in that the portion of the isopropanol or ethanol of the alcohol/water-mixture is from 65% to 85% by weight
B. mixing of the ribavirin-granulate with microcrystalline cellulose, blasting agent and highly disperse siliciumdioxide and/or talcum and/or calciumhydrogen phosphate,
C. adding a lubricant, and
D. tabletting of said mixture.

15. The method according to claim 14, further comprising the administration a cytokine.

16. The method, according to claim 3, wherein the binding agent is a polyvinylpyrrolidone with a molecular weight of less than 90,000.

17. The method, according to claim 16, wherein said binding agent is polyvidone.

18. The method, according to claim 4, wherein said sugar is milk sugar.

19. The method, according to claim 5, wherein said sieve is about 2 mm.

20. The method, according to claim 7, wherein said sieve is about 1 mm.

21. The method, according to claim 9, wherein said lubricant is selected from the group consisting of magnesium stearate, fumaric acids, adipinic acid and PEG.

22. The method, according to claim 10, wherein said blasting agent is cropovidone.

23. The method, according claim 12, wherein said film-forming agent is selected from the group consisting of hydroxypropylmethyl cellulose, hydroxypropylmethyl cellulose phthalate, ethyl cellulose, polyacrylates or shellac and derivatives thereof.

24. The method, according to claim 14, wherein said disease is a viral disease.

25. The method, according to claim 24, wherein said virus is HCV.

26. The method, according to claim 15, wherein said cytokine is interferon α and/or its derivatives.
